(19) United States
(12) Patent Application Publication
(10) Pub. No.: US 2017/0231928 A1
(43) Pub. Date: Aug. 17, 2017

(54) SOLID COMPOSITION OF FINGOLIMOD AND PREPARATION THEREOF

(71) Applicant: SUNSHINE LAKE PHARMA CO., LTD., Dongguan, Guangdong (CN)

(72) Inventors: Yi Tao, DONGGUAN (CN); Fangfang HUANG, DONGGUAN (CN); Xiaoqin WANG, DONGGUAN (CN); Jinsong YOU, DONGGUAN (CN); Lu LI, DONGGUAN (CN); Yiwen ZHONG, DONGGUAN (CN); Dexia LI, DONGGUAN (CN)

(73) Assignee: SUNSHINE LAKE PHARMA CO., LTD., Dongguan, Guangdong (CN)

(21) Appl. No.: 15/502,205

(22) PCT Filed: Aug. 21, 2015
(86) PCT No.: PCT/CN2015/087773

§ 371 Date: Feb. 7, 2017

(30) Foreign Application Priority Data
Aug. 22, 2014 (CN) 201410419236.9

(51) Int. Cl. A61K 31/137; A61K 9/16

(52) U.S. Cl. CPC A61K 31/137; A61K 9/1652; A61K 9/1694

(57) ABSTRACT

The invention provides a solid composition and preparation method thereof. The solid composition comprises fingolimod or a pharmaceutically acceptable salt thereof and a diluent, in which the diluent is complex starch. The solid composition having good compatibility of excipients, stability and dissolution can improve drug safety and increase the dissolution and absorption in vivo. The method for the preparation of the solid composition is simple to operate, low cost, and suitable for industrial production.

### CROSS-REFERENCE TO RELATED APPLICATIONS

This is a U.S. national stage application of the International Patent Application No. PCT/CN2015/087773, filed Aug. 21, 2015, which claims priority to Chinese Patent Application No. 201410419236.9, filed Aug. 22, 2014, both of which are incorporated herein by reference in their entirety.

### FIELD

The present invention relates to a solid composition of fingolimod and its preparation method thereof, and the composition has good materials compatibility.

### BACKGROUND

Fingolimod, chemically described as 2-amino-2-[2-(4-octylphenyl)ethyl]propan-1,3-diol, has the following structure:

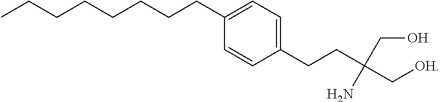 [SMILES: CCCCCCCCc1ccc(CCC(N)(CO)CO)cc1]

Fingolimod is the first novel immunosuppressant to be administered orally for the treatment of relapsing-remitting multiple sclerosis. Fingolimod capsule is launched in the United States in 2010 by Novartis under the trade name GILENYA, for the treatment of relapsing forms of multiple sclerosis (MS).

PCT Publication WO2004089341 disclosed a solid pharmaceutical composition comprising SP receptor agonist and sugar alcohol, in which the sugar alcohol is preferably mannitol. Patent application CN201310110456.9 disclosed an oral solid composition of fingolimod hydrochloride prepared by a wet granulation process comprising a diluent, a binder and a lubricant, in which the diluent is selected from lactose, pregelatinized starch, microrystalline cellulose, and a combination thereof.

### SUMMARY

According to embodiments of a first broad aspect of the present invention, there is provided a solid composition of fingolimod, which has good materials compatibility, stability and dissolution.

According to embodiments of a second broad aspect of the present invention, there is provided a method for the preparation of the solid composition, and the method is simple to operate, low cost and suitable for industrial production.

Term Definition

The term “D90” refers to the particle size of a sample of the cumulative particle size distribution reaches 90% of the grain size. It physically means the particles whose particle size is less than it account for 90%, such as “D90≦100 μm” means particles no more than 100 μm account for 90%”. D10 refers to the cumulative size distribution of a sample of 10% of the corresponding diameter. D50 refers to the total number of the particle size distribution of a sample corresponding to 50% of the particle size.

The term “Optional” or “optionally” refers to the subsequent event or situation can be but not necessarily appear. For example, “Optional any other pharmaceutical acceptable excipient” refers to other pharmaceutical acceptable excipients may be exist or not.

The term “comprising” is an open expression, which includes the contents of the specified in the present invention, but does not exclude other contents.

In the present invention, whether the term “about” is used or not, all numbers in the present invention may be approximate values. The value of each digit may differ by less than 10% or a reasonable amount which the person in the art thinks reasonable, such as differ by 1%, 2%, 3%, 4% or 5%.

### DETAILED DESCRIPTION

The inventors in the study found that fingolimod had different compatibility of different diluents, and the compatibility between complex starch as a diluent and fingolimod was best. The corresponding composition not only had good stability, but also had fast dissolution.

In one aspect, provided herein is a solid composition comprising fingolimod or a pharmaceutically acceptable salt thereof and a diluent, in which the diluent is complex starch.

The solid composition, comprises fingolimod or a pharmaceutically acceptable salt thereof, in which the pharmaceutically acceptable salt maybe inorganic salts, such as hydrochloride, hydrobromide, sulfate; or organic salts, such as acetate, fumarate, maleate, benzoate, citrate, malate, methanesulfonate and benzenesulfonate; or metal salts, such as sodium salt, potassium salt, calcium salt and aluminum salt; or ammonium salt, such as triethylamine salt; or salts with dibasic amino acids such as lysine salt.

In some embodiments, the pharmaceutically acceptable salt is fingolimod hydrochloride.

In some embodiments, the solid composition comprises 85-98 parts by weight of the complex starch, relative to 100 parts by weight of the total weight of the solid composition.

In some embodiments, the solid composition comprises flingolimod hydrochloride, and further comprises a lubricant.

In some embodiments, the lubricant is selected from magnesium stearate, calcium stearate, zinc stearate, stearic acid, fumaric acid, sodium stearoyl fumaric acid, polyethylene glycol, glycerin monobehenate, and a combination thereof.

In some embodiments, the lubricant is magnesium stearate.

In some embodiments, the solid composition comprises 0.5-10 parts by weight of a lubricant, relative to 100 parts by weight of the total weight of the solid composition.

In some embodiments, the solid composition comprises 0.5-5 parts by weight of fingolimod hydrochloride, 85-98 parts by weight of the diluent, 0.5-10 parts by weight of the lubricant, relative to 100 parts by weight of the total weight of the solid composition.

In some embodiments, the solid composition comprises 0.5-1.0 part by weight of fingolimod hydrochloride, 95-98 parts by weight of the complex starch, 1.0-2.5 parts by weight of the lubricant, relative to 100 parts by weight of the total weight of the solid composition.

In some embodiments, each unit dose of the solid composition comprises fingolimod 0.5 mg to 1.0 mg.

In some embodiments, the solid composition comprises 0.62 part by weight of fingolimod hydrochloride, 97.38 parts by weight of the complex starch, 2 parts by weight of magnesium stearate, relative to 100 parts by weight of the total weight of the solid composition.

In some embodiments, the solid composition comprises 0.59 part by weight of fingolimod hydrochloride, 97.41 parts by weight of the complex starch, 2 parts by weight of magnesium stearate, relative to 100 parts by weight of the total weight of the solid composition.

In some embodiments, the solid composition comprises 0.66 part by weight of fingolimod hydrochloride, 97.34 parts by weight of complex starch, 2 parts by weight of magnesium stearate, relative to 100 parts by weight of the total weight of the solid composition.

In some embodiments, the solid composition can be in the form of granules, and then prepared to be a capsule formulation, in addition to the above components, the solid composition further includes capsule shell, sunscreen agent and pigment.

The material of capsule shell is selected from gelatin. The sunscreen agent is selected from titanium dioxide. The pigment is selected from FD & C Red No. 3, FD & C Red No. 20, FD & C Yellow No. 6, FD & C Blue No. 2, D & C Green No. 5, D & C orange No. 5, D & C red No. 8, the caramel, red iron oxide, iron oxide yellow, and a combination thereof.

In some embodiments, the solid composition comprises fingolimod hydrochloride which has the particle size D90 not more than 100 μm. The inventors have found that, in some embodiments, fingolimod hydrochloride with particle size D90 not more than 65 μm is conducive to the solid composition to have better content uniformity and dissolution velocity.

In another aspect, provided herein is a method for preparing the solid composition. The method for preparing the solid composition comprises the steps of:

1) forming material 1: sieving the complex starch using a dry granulator, subjecting about ⅔ of the resulting product of the sieving to a first screening using a 20 mesh screen, and placing the resulting product of the first screening in a mixing bucket to obtain the material 1;

2) forming material 2: mixing fingolimod or a pharmaceutically acceptable salt thereof with the resulting product of the sieving at an amount of five folds of the fingolimod or the pharmaceutically acceptable salt, subjecting the resulting mixture to a second screening using a 40 mesh screen, subjecting the rest amount of the resulting sieving product to a third screening using a 40 mesh screen, and mixing the resulting product of the second screening and third screening with the material 1 to obtain the material 2 in the mixing bucket;

3) forming material 3: mixing the material 2 in a mixing hopper at a mixing speed of about 5 rpm to about 20 rpm for about 5 minutes to about 60 minutes to obtain the material 3;

4) forming material 4: mixing magnesium stearate with the material 3 at an amount of three-fold amount of magnesium stearate, subjecting the resulting mixing product to a fourth screening using a 40 mesh screen, and subjecting the resulting product of the fourth screening to mixing in the mixing hopper at a mixing speed of about 5 rpm to about 20 rpm for 5 minutes to 60 minutes to obtain the material 4.

In some embodiments, the method for preparing the solid composition further comprises the steps of: 5) forming products: preparing the material 4 to be a suitable dosage form to obtain the products.

In some embodiments, the material 4 is filled into capsules to obtain the products.

In some embodiments, the material 4 is prepared to tablets or granules to obtain the products.

The solid composition of fingolimod provided herein has good compatibility with excipients, stable quality; and better content uniformity, may improve the drug safety and increase the dissolution and absorption in vivo.

The method for preparing the solid composition provided herein is simple and low cost, suitable for industrial production.

### EXAMPLES

In order to make the skilled in the art to understand the present invention better, some non-limiting examples are disclosed and described in detail.

The reagents used in the present invention can be obtained from the market or be prepared by the method described in the present invention.

Comments: rpm=revolutions per minute; RRT=relative retention time; min=minute; ND=not detectable; API=fingolimod hydrochloride; DCPA=anhydrous calcium hydrogen phosphate; MCC PH200=microcrystalline cellulose PH200. SDS=Sodium Dodecyl Sulfonate; SD=standard deviation. Impurity E, F, G or H was known impurity; the peak whose RRT was 1.58, 1.66, 1.71, or 1.84 minutes was a excipient peak, was not included in the total impurities.

### Example 1

### Excipient compatibility test

1) Compatibility of API and Diluents

Some API and excipients mixture were weighed, placed in the vials, added with appropriate amount of purified water (w/w), and then sealed (simulated hot and humid environment) and placed in 60° C. oven. Sampled on the 10th day, and tested the content (%) of the related substances (single impurity and total impurities). The results were shown in Table 1.

TABLE 1
Results of compatibility between API and diluents for 10 days

| | | | After 10 days (60° C.) | | | | | | | |
|---|---|---|---|---|---|---|---|---|---|---|
| | | 0 | | | API + | API + | API + | API + | | |
| | | day | | API + | MCC | pregelatinized | complx | lactose | API + | API + |
| Sample | | API | API | DCPA | PH200 | starch | starch | monohydrate | mannitol | sucrose |
| Related | RRT = 0.55 | ND | ND | 0.09 | ND | ND | ND | 0.20 | ND | 0.15 |
| substances | RRT = 0.60 | ND | ND | 2.36 | 1.11 | 0.14 | ND | 1.78 | ND | 2.01 |
| | RRT = 0.84 | 0.06 | 0.07 | 0.07 | 0.08 | 0.14 | 0.08 | 0.09 | 0.11 | 0.16 |
| | RRT = 0.95 | ND | ND | ND | 0.05 | ND | ND | ND | 0.05 | ND |
| | RRT = 1.03 | ND | ND | 0.03 | ND | ND | ND | ND | ND | ND |
| | RRT = 1.05 | ND | ND | ND | ND | 0.09 | 0.06 | ND | ND | 0.11 |
| | RRT = 1.09 | ND | ND | 0.15 | 0.05 | ND | ND | 0.04 | ND | ND |
| | RRT = 1.28 | ND | ND | ND | 0.10 | ND | ND | ND | ND | ND |
| | RRT = 1.35 | ND | 0.03 | 0.06 | 0.04 | 0.010 | 0.06 | 0.10 | 0.11 | 0.13 |
| | RRT = 1.85 | ND | ND | ND | 0.13 | ND | ND | ND | ND | ND |
| | RRT = 2.10 | ND | ND | ND | ND | ND | ND | 0.40 | ND | 0.08 |
| | Total | 0.06 | 0.10 | 2.76 | 1.56 | 0.92 | 0.20 | 2.61 | 0.27 | 2.64 |
| | impurities ≦ 2.5% | | | | | | | | | |

As showed from Table 1, after 10 days of high temperature, the single impurity and total impurities increased, and the diluents which met the standard of total impurities included microcrystalline cellulose PH200, pregelatinized starch, complex starch and mannitol. From the amount of total impurities, it could be seen, complex starch had the best compatibility with API.

### Example 2

### Formulation and Process

| Components | Assay (%) |
|---|---|
| Fingolimod Hydrochloride | 0.62 |
| D90 = 62.168 μm | |
| Complex starch | 97.38 |
| Magnesium stearate | 2 |

Batch: 12000 pills

Preparation Procedure:
I) forming material 1: U5 dry granulator was used to sieve complex starch (rotational speed was 1440 rpm), about ⅓ prescribed amount of complex starch was kept out; the rest of the complex starch was sieved through a 20 mesh screen, and then it was placed in a mixing bucket to obtain material 1;
II) forming material 2: prescribed amount of fingolimod hydrochloride and its five-fold amount of complex starch by weight was mixed, followed with sieving through a 40 mesh screen and then was put into the mixing bucket containing the material 1; all the rest of the complex starch was shock through a 40 mesh screen and then was put into the mixing bucket containing material 1 to obtain material 2;
III) forming material 3: the material 2 was mixed in a mixing hopper for 5 minutes to 60 minutes to obtain material 3, in which the rotational speed was from 5 rpm to 20 rpm;
IV) forming material 4: prescribed amount of magnesium stearate and its three-fold amount of material 3 was mixed by hand, then shock through a 40 mesh screen, added into a mixing hopper, and mixed for 5 minutes to 60 minutes to obtain material 4, in which the rotational speed was from 5 rpm to 20 rpm;
V) filling into capsules: NJP-500 automatic capsule filling machine was used to fill the material 4 into No. 3 gelatin hollow capsules, and the weight of the contents of each capsule was about 90 mg.

### Example 3

### Formulation and Process

| Components | Assay (%) |
|---|---|
| Fingolimod Hydrochloride | 0.59 |
| D90 = 62.168 μm | |
| Complex starch | 97.41 |
| Magnesium stearate | 2 |

Batch: 15000 pills

The preparation process was same as in Example 2.

### Example 4

### Formulation and Process

| Components | Assay (%) |
|---|---|
| Fingolimod Hydrochloride | 0.66 |
| D90 = 62.168 μm | |
| Complex starch | 97.34 |
| Magnesium stearate | 2 |

Batch: 10000 pills

The preparation process was same as in Example 2.

### Example 5

### Formulation and Process

| Components | Assay (%) |
|---|---|
| Fingolimod Hydrochloride | 0.62 |
| D90 = 62.168 μm | |
| Complex starch | 97.88 |
| Magnesium stearate | 1.5 |

Batch: 12000 pills

The preparation process was same as in Example 2.

### Example 6

### Formulation and Process

| Components | Assay (%) |
|---|---|
| Fingolimod Hydrochloride | 0.56 |
| D90 = 62.168 μm | |
| Complex starch | 87.64 |
| Magnesium stearate | 1.8 |

Batch: 12000 pills

The preparation process was same as in Example 2.

### Example 7

### Formulation and Process

| Components | Assay (%) |
|---|---|
| Fingolimod Hydrochloride | 0.62 |
| D90 = 62.168 μm | |
| Complex starch | 96.88 |
| Magnesium stearate | 2.5 |

Batch: 12000 pills

The preparation process was same as in Example 2.

### Example 8

### Stability Test

1) The Related Substance and the Contents in Accelerated Stability Test

The experimental method was conducted according to the design guidelines for drug stability test in the appendix XIXC of Chinese Pharmacopeia (the second part, 2010 Edition).

Detection method: the samples prepared in Example 2 were placed at 40° C. and 75% RH (Relative Humidity) condition for 6 months, sampled at the end of the first, the third, and the sixth month to test the contents and the related substances. The test method for the related substance(single impurity and total impurities) was HPLC, which was used to related substances. The results were showed in Table 2.

The test condition of HPLC was:

Chromatographic column: Waters Xterra™ MS C8; 4.6×50 mm, 2.5 μm;

Flow rate: 1.5 mL/min;

Column temperature: 30° C.;

Sample disc temperature: 8° C.;

Determine wavelength: 215 nm;

Injection volume: 10 μL;

Running time: 35 min;

Moving phase: phase A (pH2.6 Buffer:methanol=93:7) and phase B (acetonitrile);

The sample was gradiently eluted, and the elution program was shown as follows:

| Time (min) | Phase A (%) | Phase B (%) |
|---|---|---|
| 0→1 | 70 | 30 |
| 1→15 | 70→58 | 30→42 |
| 15→28 | 58→5 | 42→95 |
| 28→30 | 5 | 95 |
| 30→30.1 | 5→70 | 95→30 |
| 30.1→35 | 70 | 30 |

TABLE 2
The contents and related substances detecting results

| Samples: Example 2 | | 0 day | First month | Third month | Sixth month | Standard |
|---|---|---|---|---|---|---|
| | Impurity/ | Impurity | Impurity | Impurity | Impurity | Limit |
| | RRT | (%) | (%) | (%) | (%) | (%) |
| Related | E | ND | ND | ND | ND | 1.0 |
| substances | F | ND | ND | ND | ND | 1.0 |
| | G | ND | ND | ND | ND | 1.0 |
| | H | ND | ND | ND | ND | 1.0 |
| | 1.58 | ND | 0.11 | ND | ND | ND |
| | 1.66 | 0.25 | 0.34 | 0.29 | 0.17 | ND |
| | 1.71 | 0.61 | 0.46 | 0.57 | 0.38 | ND |
| | 1.84 | 0.83 | 0.88 | 0.85 | 0.68 | ND |
| | Total | 0.00 | 0.00 | 0.00 | 0.00 | 2.5 |
| | Impurities | | | | | |

| | | | | | |
|---|---|---|---|---|---|
| Assay | 101.1 | 99.0 | 96.9 | 98.8 | Between 90.0% |
| | | | | | to 110.0% of the |
| | | | | | mark amount |

2) Dissolving experiment

Six samples of Example 2 were placed for six months and the dissolution was tested at the 0 day, and the end of the first, third, and sixth month. The experimental method was conducted according to the first dissolution determination method in the appendix XC of Chinese Pharmacopeia (2010 Edition). The results were showed in Table 3.

TABLE 3
Results of dissolution rate (0.1 mol/L HCl+
0.2% SDS, 500 mL, basket-rotated method, 100 rpm)

| | Acceleration | Disslution (mean ± SD, %, n = 6) | | | | | |
|---|---|---|---|---|---|---|---|
| Samples | time | 5 min | 10 min | 15 min | 20 min | 30 min | 45 min |
| Example 2 | 0 day | 73.5 ± 5.5 | 86.9 ± 3.2 | 90.7 ± 2.7 | 92.9 ± 2.4 | 95.9 ± 1.9 | 97.4 ± 1.9 |
| | 1 month | 70.0 ± 3.6 | 84.4 ± 1.9 | 89.9 ± 1.4 | 91.2 ± 1.3 | 91.9 ± 2.5 | 93.7 ± 2.7 |
| | 3 months | 71.3 ± 10.2 | 87.9 ± 2.9 | 92.0 ± 3.2 | 94.3 ± 3.0 | 96.2 ± 3.3 | 97.9 ± 3.1 |
| | 6 months | 71.7 ± 4.7 | 85.3 ± 2.5 | 90.1 ± 3.0 | 92.7 ± 2.7 | 95.5 ± 3.1 | 96.3 ± 3.2 |

From the results of Table 2 and Table 3, it could be seen, accelerated conditions six months, the related substances of the formulation had no increasing trend, and had no total impurities. After 6 months, the dissolution rate was same as 0 day. The solid composition provided herein had good stability and dissolution rate.

Some embodiments of the invention are disclosed herein, obviously, a skilled artisan can make any alterations, changes or combinations thereof appropriately to implement and apply the present invention without departing from the content, spirit and scope of the present invention. The skilled in the art can learn from the present invention and improve the process parameters appropriately. It should be noted that it can be readily apparent to those of ordinary skill in the art that certain modifications may be made thereto within the scope of the invention.

1. A solid composition, comprising fingolimod or a pharmaceutically acceptable salt thereof and a diluent, wherein the diluent is complex starch.

2. The solid composition according to claim 1, wherein the pharmaceutically acceptable salt is fingolimod hydrochloride.

3. The solid composition according to claim 1, comprising 85-98 parts by weight of the complex starch, relative to 100 parts by weight of the total weight of the solid composition.

4. The solid composition according to claim 1, comprising 0.5-10 parts by weight of a lubricant, relative to 100 parts by weight of the total weight of the solid composition.

5. The solid composition according to claim 4, wherein the lubricant is selected from magnesium stearate, calcium stearate, zinc stearate, stearic acid, fumaric acid, sodium stearoyl fumaric acid, polyethylene glycol, glycerin monobehenate, and a combination thereof

6. The solid composition according to claim 1, comprising 0.5-5 parts by weight of fingolimod hydrochloride, 85-98 parts by weight of the diluent, 0.5-10 parts by weight of the lubricant, relative to 100 parts by weight of the total weight of the solid composition.

7. The solid composition according to claim 1, comprising 0.5-1.0 part by weight of fingolimod hydrochloride, 95-98 parts by weight of the complex starch, 1.0-2.5 parts by weight of the lubricant, relative to 100 parts by weight of the total weight of the solid composition.

8. The solid composition according to claim 1, comprising 0.5 mg to 1.0 mg of fingolimod in each unit dose.

9. The solid composition according to claim 1, comprising 0.62 part by weight of fingolimod hydrochloride, 97.38 parts by weight of the complex starch, 2 parts by weight of magnesium stearate, relative to 100 parts by weight of the total weight of the solid composition.

10. The solid composition according to claim 1, comprising 0.66 part by weight of fingolimod hydrochloride, 97.34 parts by weight of the complex starch, 2 parts by weight of magnesium stearate, relative to 100 parts by weight of the total weight of the solid composition.

11. The solid composition according to claim 1, comprising 0.59 part by weight of fingolimod hydrochloride, 97.41 parts by weight of the complex starch, 2 parts by weight of magnesium stearate, relative to 100 parts by weight of the total weight of the solid composition.

12. The solid composition according to claim 2any one of claim 2, wherein the particle size D90 of fingolimod hydrochloride is not more than 100 μm.

13. The solid composition according to claim 1, the dosage form of the solid composition is granules or capsules.

14. A method for preparing the solid composition according to claim 1, comprising the steps of:
(1) forming material 1: sieving the complex starch using a dry granulator,subjecting about ⅔ of the resulting product of the sieving to a first screening using a 20 mesh screen, and placing the resulting product of the first screening in a mixing bucket to obtain the material 1;
(2) forming material 2: mixing fingolimod or a pharmaceutically acceptable salt thereof with the resulting product of the sieving at an amount of five folds of the fingolimod or the pharmaceutically acceptable salt, subjecting the resulting mixture to a second screening using a 40 mesh screen, subjecting the rest amount of the resulting sieving product to a third screening using a 40 mesh screen, and mixing the resulting product of the second screening and third screening with the material 1 to obtain the material 2 in the mixing bucket;
(3) forming material 3: mixing the material 2 in a mixing hopper at a mixing speed of about 5 rpm to about 20 rpm for about 5 minutes to about 60 minutes to obtain the material 3;
(4) forming material 4: mixing magnesium stearate with the material 3 at an amount of three-fold amount of magnesium stearate, subjecting the resulting mixing product to a fourth screening using a 40 mesh screen, and subjecting the resulting product of the fourth screening to mixing in the mixing hopper at a mixing speed of about 5 rpm to about 20 rpm for 5 minutes to 60 minutes to obtain the material 4.

15. The solid composition according to claim 6, wherein the particle size D90 of fingolimod hydrochloride is not more than 100 μm.

16. The solid composition according to claim 7, wherein the particle size D90 of fingolimod hydrochloride is not more than 100 μm.

17. The solid composition according to claim 2, the dosage form of the solid composition is granules or capsules.

18. The solid composition according to claim 3, the dosage form of the solid composition is granules or capsules.

19. The solid composition according to claim 7, the dosage form of the solid composition is granules or capsules.

20. The solid composition according to claim 8, the dosage form of the solid composition is granules or capsules.
